(19) United States
(12) Patent Application Publication
(10) Pub. No.: US 2009/0286795 A1
(43) Pub. Date: Nov. 19, 2009

(54) USE OF VASCULAR ENDOTHELIAL GROWTH FACTOR RECEPTOR INHIBITORS FOR THE TREATMENT OF CANCER

(76) Inventor: Amanda J. Littlewood-Evans, Oberwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/302,578

(22) PCT Filed: May 31, 2007
(86) PCT No.: PCT/EP2007/004827

§ 371 Date: Nov. 26, 2008

(30) Foreign Application Priority Data
Jun. 2, 2006 (GB) 0610925.0

(51) Int. Cl. A61K 31/50; C12Q 1/32

(52) U.S. Cl. 514/248; 435/26

(57) ABSTRACT

The invention relates to a method for screening patients for tumor burden and the use of VEGF-R inhibitors alone or in combination with chemotherapy for the treatment of gastrointestinal, genitourinary, lymphoid and pulmonary (small cell and non-small cell) cancer patients and patients with cancers of neural crest origin having high serum or plasma LDH5 levels.

The invention relates to a method for screening patients for tumor burden and the use of VEGF-R inhibitors alone or in combination with chemotherapy for the treatment of gastrointestinal, genitourinary, lymphoid and pulmonary (small cell and non-small cell) cancer patients and patients with cancers of neural crest origin having high serum or plasma LDH5 levels.

The use of Vascular Endothelial Growth Factor Receptor (VEGF-R) inhibitors for the treatment of proliferative diseases is already known in the art. At the centre of the network regulating the growth and differentiation of the vascular system and its components, both during embryonic development and normal growth and in a wide number of pathological anomalies and diseases, lies the angiogenic factor known as “Vascular Endothelial Growth Factor”, along with its cellular receptors (see Breier, G., et al., Trends in Cell Biology 6, 454-6 [1996] and references cited therein). VEGF is a dimeric, disulfide-linked 46-kDa glycoprotein. VEGF receptors are transmembranous receptor tyrosine kinases. They are characterized by an extracellular domain with seven immunoglobulin-like domains and an intracellular tyrosine kinase domain. Certain diseases are known to be associated with deregulated angiogenesis, for example diseases caused by ocular neovascularisation, such as retinopathies, age-related macula degeneration, psoriasis, arteriosclerosis and especially proliferative diseases, for example so-called solid tumours such as colorectal cancer and liquid tumours such as leukaemia. A large number of compounds inhibiting the VEGF-R tyrosine kinase activity has been described in the art.

Human cancer patients displaying certain prognostic factors upon diagnosis are considered as patients having a lower expected survival time compared to those patients lacking such prognostic factors. Prognostic factors for survival have, for instance, been identified for colorectal cancer suitable to determine the status of the disease and to predict the expected survival time of the patient (N. Kemeny et al, Prognostic variables in patients with hepatic metastases from colorectal cancer, Cancer 63(4), 1989, 742-7).

High serum total LDH (lactase dehydrogenase) levels have been associated with poor prognosis in many solid tumors, including lung cancer¹⁻³, pancreatic cancer⁴, and colorectal cancer⁵⁻⁶. This serum total LDH appears to be a surrogate marker for tumors that are bulky, hypoxic, and aggressive and that favor metastatic growth⁵.

Blood vessels supply the tumor with oxygen and nutrients. As a tumor grows in size and cell density, it reaches the limits of oxygen and energy diffusion. At a distance of approximately 100 micron from the blood vessel, the tumor cells become hypoxic and genes associated with hypoxia such as Hypoxia Inducible Factors, HIFs, become stabilized. These subsequently bind to hypoxia response element (HRE) in the promoter regions of various genes including LDHA and increase production of the protein.

LDH (lactase dehydrogenase) is a glycolytic enzyme composed of 5 tetrameric isoforms—LDH1-5 which are composed of M and H subunits.

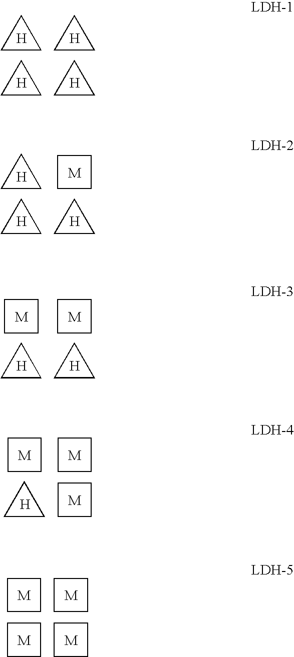

The H subunit is derived from the LDHB gene and is not regulated by hypoxia. The M subunit is derived from the LDHA gene and is increased during hypoxia.

In colorectal adenocarcinomas and lung carcinomas, serum total LDH correlates with high tumor tissue LDH-5 and markers of hypoxia in the tumor^{3,7}. Thus tumor LDH-5 can be thought of as an endogenous marker of hypoxia⁸. Furthermore, it has been shown that in adjacent normal and tumerous tissue from lung and bladder, there is a shift from the LDH-H subunit to the M containing isoforms⁹⁻¹¹.

Surprisingly, it has now been found that LDH5 from plasma or serum is likely to be tumor derived and is a more specific marker of a hypoxic tumor than total LDH.

High LDH5 in plasma or serum correlates to tumor burden and can therefore be used as a stratification marker to identify those patients who have the highest tumor burden and are therefore more responsive to therapy with VEGF-R inhibitors. Since the LDH5 levels predict tumor burden, serum or plasma LDH5 can be used as a non invasive method to monitor response to therapy with VEGF-R inhibitors.

In the studies outlined in the experimental part below, a tight correlation of plasma or serum LDH5 to tumor burden from 2 different tumor types is shown. This correlation is likely to be applicable across many tumor types. This correlation could then be used as a biomarker for instance in clinical trials to assess tumor response or relapse from a particular therapy. It should be applicable to all types of therapy from anti tumor to anti angiogenic therapy.

Hence, the present invention relates to a non invasive method for screening patients for tumor burden by measuring serum or plasma LDH5 for selecting patients for successful therapy with a VEGF-R inhibitor, high serum or plasma LDH5 being a criteria for selecting candidates.

The present invention further relates to non invasive method for screening patients for tumor burden by measuring serum or plasma LDH5 as a surrogate or biomarker of tumor burden for monitoring response/relapse to therapy with a VEGF-R inhibitor.

The present invention further relates to the use of VEGF-R inhibitors alone or in combination with chemotherapy for the treatment of gastrointestinal, genitourinary, lymphoid and pulmonary (small cell and non-small cell) cancer patients having high serum or plasma LDH5 levels and patients with cancers of neural crest origin having high serum or plasma LDH5 levels.

The present invention further relates to the use of LDH5 in plasma or serum as a biomarker to monitor tumor size during therapy with anti VEGF-R inhibitors which could then be used to assess tumor progression, stabilization or regression.

The present invention further relates to the use of VEGF-R inhibitors alone or in a combination as mentioned above for the preparation of medicaments for the treatment of gastrointestinal, genitourinary, lymphoid and pulmonary (small cell and non-small cell) cancer patients having high serum or plasma LDH5 levels, and patients with cancers of neural crest cell origin having high serum or plasma LDH5 levels, a commercial package or product comprising such a combination as a combined preparation for simultaneous, separate or sequential use together with instructions to use such combination in the treatment of gastrointestinal, genitourinary, lymphoid and pulmonary (small cell and non-small cell) cancer patients having high serum or plasma LDH5 levels and patients with cancers of neural crest cell origin having high serum or plasma LDH5 levels, and to a method of treating gastrointestinal, genitourinary, lymphoid and pulmonary (small cell and non-small cell) cancer patients having high serum or plasma LDH5 levels and patients having cancer of neural crest cell origin having high serum or plasma LDH5 levels.

More specifically, the present invention relates to
a method of treating gastrointestinal, genitourinary, lymphoid or pulmonary (small cell and non-small cell) cancer comprising administering a therapeutically effective amount of a VEGF-R inhibitor to a human patient having high serum or plasma LDH5 levels; and to
a method of treating gastrointestinal, genitourinary, lymphoid or pulmonary (small cell and non-small cell) cancer or patients having cancer of neural crest cell origin comprising administering a therapeutically effective amount of a 4-pyridylmethyl-phthalazine derivative to a human patient having high serum or plasma LDH5 levels,
A method to screen patients for serum or plasma LDH5 levels before start of therapy, and to monitor levels throughout therapy as a surrogate or biomarker marker of tumor burden—thus giving information on response/relapse to therapy with a VEGF-R inhibitor (anti VEGFR therapy).

The term “gastrointestinal, genitourinary, lymphoid, and pulmonary (small cell and non-small cell) cancer and cancer of neural crest cell origin” as used herein relates in particular to colorectal cancer, lung cancer, such as non-small cell lung cancer and small cell lung cancer, melanoma, pheochromocytoma, neuroblastoma, pancreatic cancer, lymphoma, especially Burkitt, Hodgkins and Non-Hodgkins lymphoma, testicular cancer, mesothelioma, renal cell carcinoma, ovarian cancer and prostate cancer. In one preferred embodiment of the present invention the cancer type is colorectal cancer, especially metastatic colorectal cancer.

LDH5 values can be determined from plasma samples of the patients by standard laboratory serum evaluations known in the art. For that purpose, typically, blood samples (between 2 and 10 mL) is drawn from a vein or from a heel, finger, toe, or earlobe and collected into pre-cooled (ice bath) heparinized tubes, gently inverted 8 to 10 times and immediately placed into an ice bath. Within 30 minutes, plasma is prepared by centrifugation (e.g. ca. 2,000×g, 4° C., 10 min). Following centrifugation, the plasma sample can be stored frozen at ≦−18° C. until analysis.

LDH5 values can be determined from serum samples of the patients by standard laboratory serum evaluations known in the art. For that purpose, typically, blood samples (between 2 and 10 mL) is drawn from a vein or from a heel, finger, toe, or earlobe and collected into normal microtubes containing Z-Gel to allow a better separation of the blood layers (Sarstedt, Nümbrecht, Germany). This is left at room temperature for 30 minutes. Within 30 minutes, serum is prepared by centrifugation (e.g. ca. 2,000×g, 4° C., 10 min). Following centrifugation, the serum sample can be stored frozen at ≦−18° C. until analysis.

In a broader aspect, the present invention relates to the use of compounds which decrease by any kind of mechanism the activity of the VEGF in gastrointestinal, genitourinary, lymphoid and pulmonary (small cell and non-small cell) cancer patients with poor prognosis and patients having cancer of neural crest cell origin with poor prognosis, especially patients having high serum or plasma LDH5 levels. Compounds which decrease the activity of the VEGF are especially compounds which inhibit the VEGF receptor tyrosine kinase, but also compounds which inhibit a VEGF receptor and compounds binding to VEGF, and are in particular those compounds, proteins and monoclonal antibodies generically and specifically disclosed in WO 98/35958, WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285; in each case in particular in the compound claims and the final products of the working examples, the subject-matter of the final products, the pharmaceutical preparations and claims is hereby incorporated into the present application by reference to these publications. The compounds used as active ingredients alone or in the combinations disclosed herein can be prepared and administered as described in the cited documents, respectively.

In one embodiment, the present invention relates to the use of 4-pyridylmethyl-phthalazine derivatives alone or in combination with chemotherapy by administering agents contemporaneously, separately or sequentially to treat gastrointestinal, genitourinary, lymphoid or pulmonary (small cell and non-small cell) cancer or a cancer of neural crest cell origin in patients having high serum or plasma LDH5 levels.

4-Pyridylmethyl-phthalazine derivatives being inhibitors of VEGF receptor tyrosine kinase and their preparation, pharmaceutical formulations thereof and methods of making such compounds are described in WO00/59509, EP02/04892, WO01/10859 and, in particular, in U.S. Pat. No. 6,258,812, which are here incorporated by reference. Most preferred are the compounds of formula I

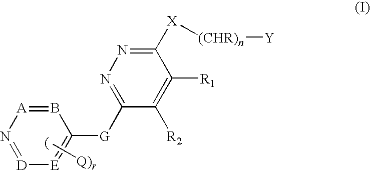

wherein
r is 0 to 2,
n is 0 to 2,
m is 0 to 4,
R₁ and R₂ (i) are lower alkyl or
(ii) together form a bridge in subformula I*

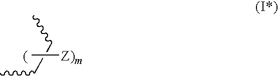

the binding being achieved via the two terminal carbon atoms, or
(iii) together form a bridge in subformula I**

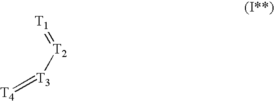

wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein one or more N atoms carry an oxygen atom; with the stipulation that, if Y is pyridyl or unsubstituted cycloalkyl, X is imino, and the remaining radicals are as defined, G is selected from the group comprising lower alkylene, —CH₂—O—, —CH₂—S—, oxa and thia;
and their pharmaceutically acceptable salts.

The radicals and symbols as used in the definition of a compound of formula I have the meanings as disclosed in WO 98/35958 which publication is hereby incorporated into the present application by reference.

The term “PTK787” or “PTK/ZK” or “PTK787/ZK222584” as used herein means a VEGF receptor tyrosine inhibitor of formula I wherein r, n and m are each 0, R₁ and R₂ together form a bridge of subformula I*, A, B, D and E are each CH, G is methylene, X is imino, Y is 4-chlorophenyl, and the bonds characterized by a wavy line are double bonds. Such VEGF receptor tyrosine inhibitor of formula I is very preferred for the present invention. Most preferably, PTK787 is employed in the form of its succinate salt.

Studies in humans have shown PTK787 to be well tolerated and to reduce tumor vascular permeability. It is understood that further references to PTK787 are intended to include pharmaceutically acceptable salts thereof.

In one embodiment of the present invention, the activity of the VEGF is decreased by administration of “AVASTIN”®.

Chemotherapy for the treatment of proliferative diseases is known in the art. When applied in combination with chemotherapy, the agents are administered contemporaneously, separately or sequentially.

In a preferred embodiment of the present invention, the chemotherapy comprises a platinum compound and/or an antineoplastic antimetabolite and, optionally, folinic acid. In a specific embodiment of the present invention, the chemotherapy comprises a platinum compound, 5-fluorouracil and folinic acid. In a further specific embodiment of the present invention, the chemotherapy comprises a platinum compound, capecitabine and folinic acid.

In another embodiment of the present invention, the chemotherapy comprises a topoisomerase I inhibitor and/or an antineoplastic antimetabolite and, optionally, folinic acid. In a specific embodiment of the invention, the chemotherapy comprises a topoisomerase I inhibitor, 5-fluorouracil or capecitabine, and folinic acid.

The term “antineoplastic antimetabolite” includes, but is not limited to, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

5-Fluorouracil can be prepared, e.g., as described in U.S. Pat. No. 2,802,005. It can be employed in the present invention as marketed, e.g., under the trademark EFUDEX™, FLURACIL™ or FLUROBLASTIN™. Tegafur can be employed especially in the form of a composition as disclosed in U.S. Pat. No. 5,116,600 and U.S. Pat. No. 5,525,603. Furthermore, tegafur can be administered, e.g., in the form as it is marketed under the trademarks FTORAFUR™, LAMAR™ or NEBEREK™. Capecitabine can be administered, e.g., in the form as disclosed in U.S. Pat. No. 5,472,949 or in the form as it is marketed, e.g., under the trademark XELODA™. Cladribine can be prepared, e.g., as disclosed in U.S. Pat. No. 4,760,135. It can be administered, e.g., in the form as it is marketed under the trademarks LEUSTATIN™ or LEUSTAT™. Cytarabine can, e.g., be prepared as disclosed in U.S. Pat. No. 3,116,282 or by Hessler in J. Org. Chem. 41 (1970) 1828. It can be administered, e.g., in the form as it is marketed under the trademarks ARA-C™, CYTOSAR™ or UDICIL™. A suitable salt of such compound is cytarabine ocfosfate (STARASID™) which can be prepared as described in U.S. Pat. No. 4,812,560. Fludarabine phosphate can be prepared as described in U.S. Pat. No. 4,357,324. It can be applied as marketed under the trademark FLUDARA™. Gemcitabine can be administered, e.g., in accordance with the disclosure of U.S. Pat. No. 5,464,826 or in the form as it is marketed, e.g., under the trademark GEMZAR™. 6-Mercaptopurine (6-purinethiol) can, e.g., be prepared as disclosed in U.S. Pat. No. 2,933,498. It can be employed as marketed, e.g., under the trademark LEUKERIN™ or PURINETHOL™. Hydroxyurea can, e.g., be prepared as disclosed in U.S. Pat. No. 2,705,727. Methotrexate can be employed as marketed, e.g., under the trademark FOLEX™ or MTX™. Edatrexate can, e.g., be prepared as disclosed in U.S. Pat. No. 4,369,319.

The term “folinic acid” relates to “N-[4-[[(2-amino-5-formyl-1,4,5,6,7,8-hexahydro-4-oxo-6-pteridinyl)methyl]amino]benzoyl-L-glutamic acid, which is marketed, e.g., under the trademark LEUCOVORIN™.

The term “platinum compound” as used herein means carboplatin, cisplatin or oxaliplatin. Preferably, the platinum compound is oxaliplatin.

The term “carboplatin” as used herein relates to the antineoplastic agent cis-diamine (1,1-cyclobutane dicarboxylato) platinum(II), which is disclosed, e.g., in U.S. Pat. No. 4,140,707 or by R. C. Harrison et al. in Inorg. Chim. Acta 46, L15 (1980). This drug can be administered e.g., in the form as it is marketed, e.g. under the trademark CARBOPLAT™ or PARAPLATIN™.

The term “oxaliplatin” as used herein relates to the antineoplastic agent also known as oxalatoplatinum, which is disclosed, e.g., in U.S. Pat. No. 5,716,988. This drug can be administered e.g., in the form as described in the cited US patent or in the form it is marketed, e.g. under the trademark ELOXANTINE™ or 1-OHP™.

The term “cisplatin” as used herein relates to the antineoplastic agent also known as cis-diaminedichloroplatinum, which compound and its use as antineoplastic agent is disclosed, e.g., in DE 2,318,020.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

In a broader sense of the invention, the term “chemotherapy” refers to the administration of an antineoplastic agent selected from the group that includes, but is not limited to topoisomerase II inhibitors, microtubule active agents, protein kinase C inhibitors, gonadorelin agonists, anti-androgens, bisphosphonates, histone deacetylase inhibitors, S-adenosylmethionine decarboxylase inhibitors, and trastuzumab.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “microtubule active agents” relates to microtubule stabilizing and microtubule destabilizing agents selected from the group consisting of paclitaxel, docetaxel, eleutherobin, the vinca alkaloids, e.g., vinblastine, especially vinblastine sulfate, vincristine especially vincristine sulfate, and vinorelbine and discodermolide. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™.

The term “protein kinase C inhibitors”, refers in particular to staurosporine derivatives, and preferably to those disclosed in U.S. Pat. No. 5,093,330. Such compounds can be administered in the form as disclosed in WO99/48896.

The term “anti-angiogenic compounds” as used herein relates to thalidomide (THALOMID™) SU5416, sorafenib, sutent.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™. Abarelix can be formulated, e.g. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

The term “histone deacetylase inhibitors” as used herein includes, but is not limited to MS-275, SAHA, FK228 (formerly FR901228), Trichostatin A and the compounds disclosed in WO 02/22577, in particular NVP-LAQ824 or its lactate salt and NVP-LBH589.

The term “S-adenosylmethionine decarboxylase inhibitors” as used herein includes, but is not limited to the compounds disclosed in U.S. Pat. No. 5,461,076.

“Trastuzumab” can be administered, e.g., in the form as it is marketed, e.g. under the trademark HERCEPTIN™.

The structure of the active agents identified by code nos., generic or trade names may be taken from the actual edition of the standard compendium “The Merck Index” or from databases, e.g. Patents International or other IMS World Publications. The corresponding content thereof is hereby incorporated by reference.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Thus, the present invention further includes a commercial package comprising 1-(4-chloroanilino)-4-(4-pyridylmethyl)phthalazine or a pharmaceutically acceptable salt thereof in a form suitable for oral administration and instructions to administer the 1-(4-chloroanilino)-4-(4-pyridylmethyl)phthalazine or a pharmaceutically acceptable salt thereof to a gastrointestinal, genitourinary, lymphoid or pulmonary (small cell and non-small cell) cancer patients having high serum or plasma LDH5 levels or patients having cancer of neural crest cell origin having high serum or plasma LDH5 levels.

The present invention also relates to the use of a combination as disclosed herein for the treatment of gastrointestinal, genitourinary, lymphoid or pulmonary (small cell and non-small cell) cancer or a cancer of neural crest cell origin in a patient having high serum or plasma LDH5 levels and for the preparation of a medicament for the treatment of gastrointestinal, genitourinary, lymphoid or pulmonary (small cell and non-small cell) cancer or a cancer of neural crest cell origin characterized by high serum or plasma LDH5 levels.

The term “metastatic growth” as used herein comprises the metastatic spread of tumors and the growth and development of micrometastases in other organs of the cancer patients.

It will be understood that references to the combination partners (a) and (b) are meant to also include the pharmaceutically acceptable salts of the compounds.

In general, for the treatment of colorectal cancer, the 4-pyridylmethyl-phthalazine derivative can be given orally on a continuous basis, for example once daily. For 1-(4-chloroanilino)-4-(4-pyridylmethyl)phthalazines succinate, a daily oral dose in the range from 750 mg to 2500 mg, especially in the range from 1000 mg to 1500 mg/day, more preferably in the range from 1200 mg to 1300 mg/day, most preferably 1250 mg/day, are contemplated as a pharmaceutically effective dose. However, other administration schedules are also likely to be effective and are included within the scope of the present invention.

When the combination partners employed in the combinations as disclosed herein are applied in the form as marketed as single drugs, their dosage and mode of administration can take place in accordance with the information provided on the package insert of the respective marketed drug in order to result in the beneficial effect described herein, if not mentioned herein otherwise.

The chemotherapy is generally administered according to established administration regimen. Such administration regimens, for example the deGramont regimen for colorectal cancer, are known in the art (A De Gramont et al, J. Clin. Onc. 18(16), 2000, 293847). In a specific embodiment, the chemotherapy comprises the administration of oxaliplatin, folinic acid and 5-fluorouracil according to an established administration regimen, such as those known in the art. A particular chemotherapy regimen whereby 85 mg/m² of oxaplatin is administered on day 1,200 mg/m² of folinic acid is given as a 2 hour infusion on days 1 and 2, and 5-fluorouracil is administered as a bolus at a dose of 400 mg/m² followed by 600 mg/m² over 22 hours on days 1 and 2 and is given every 14 days is particularly useful.

5-Fluorouracil may be administered to a human in a dosage range varying from about 50 to 1000 mg/m² day, e.g. 500 mg/m² day.

Capecitabine may be administered to a human in a dosage range varying from about 10 to 1000 mg/m² day.

Gemcitabine hydrochloride may be administered to a human in a dosage range varying from 10 to about 1000 mg/week.

Methotrexate may be administered to a human in a dosage range varying from about 5 to 500 mg/m² day.

ZD 1694 (RALTITREXED™) can be administered to a human in a dosage range varying from about 2.0 to 4.0 mg/m², e.g., 3.5 mg/m², every 3 weeks as a 15 minute infusion.

Carboplatin may be administered intravenously to a human in a dosage range varying from about 100 to 400, e.g. 200, mg/m² body surface about every four to six weeks.

Oxaliplatin may be administered intravenously to a human in a dosage range varying from about 25 to 135, e.g. 45 or 85, mg/m² body surface about every two to three weeks.

Cisplatin may be administered to a human in a dosage range varying from about 25 to 100 mg/m² about every three weeks.

Topotecan may be administered to a human in a dosage range varying from about 1 to 5 mg/m² day.

Irinotecan may be administered to a human in a dosage range varying from about 50 to 350 mg/m² day.

Vinblastine may be administered to a human in a dosage range varying from about 1.5 to 10 mg/m² day. Vincristine sulfate may be administered parenterally to a human in a dosage range varying from about 0.025 to 0.05 mg/kg body weight*week. Vinorelbine may be administered to a human in a dosage range varying from about 10 to 50 mg/m² day. Etoposide phosphate may be administered to a human in a dosage range varying from about 25 to 115 mg/m² day, e.g. 56.8 or 113.6 mg/m² day. Teniposide may be administered to a human in a dosage range varying from about 75 to 150 mg about every two weeks. Doxorubicin may be administered to a human in a dosage range varying from about 10 to 100 mg/m² day, e.g. 25 or 50 mg/m² day. Epirubicin may be administered to a human in a dosage range varying from about 10 to 200 mg/m² day. Idarubicin may be administered to a human in a dosage range varying from about 0.5 to 50 mg/m² day. Mitoxantrone may be administered to a human in a dosage range varying from about 2.5 to 25 mg/m² day. Paclitaxel may be administered to a human in a dosage range varying from about 50 to 300 mg/m² day. Alendronic acid may be administered to a human in a dosage range varying from about 5 to 10 mg/day. Clodronic acid may be administered to a human e.g. in a dosage range varying from about 750 to 1500 mg/day. Etridonic acid may be administered to a human in a dosage range varying from about 200 to 400 mg/day. Ibandronic acid may be administered to a human in a dosage range varying from about 1 to 4 mg every three to four weeks. Risedronic acid may be administered to a human in a dosage range varying from about 20 to 30 mg/day. Pamidronic acid may be administered to a human in a dosage range varying from about 15 to 90 mg every three to four weeks. Tiludronic acid may be administered to a human in a dosage range varying from about 200 to 400 mg/day. Trastuzumab may be administered to a human in a dosage range varying from about 1 to 4 mg/m² week. Bicalutamide may be administered to a human in a dosage range varying from about 25 to 50 mg/m² day.

It is one objective of this invention to provide a pharmaceutical composition comprising a quantity, which is jointly therapeutically effective against a proliferative disease comprising a combination as described herein. In this composition, the combination partners (a) and (b) can be administered together, one after the other or separately in one combined unit dosage form or in two separate unit dosage forms. The unit dosage form may also be a fixed combination.

The pharmaceutical compositions for separate administration of the combination partners (a) and (b) and for the administration in a fixed combination, i.e. a single pharmaceutical composition comprising at least two combination partners (a) and (b), according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

It can be shown by established test models that VEGF-R inhibitors alone or in combination with chemotherapy result in the beneficial effects described herein before. The person skilled in the pertinent art is fully enabled to select a relevant test model to prove such beneficial effects. The pharmacological activity may, for example, be demonstrated in a clinical study or in a test procedure as essentially described hereinafter.

Suitable clinical studies are, for example, randomized, double-blind, placebo-controlled, parallel studies in patients with metastatic colorectal cancer, but also dose escalation studies. For treatment schemes including chemotherapy, optionally a standard antiemetic regimen for the prophylaxis of acute emesis can be given to the patient on the day of chemotherapy, e.g. a 5HT₃ antagonist such as granisetron or ondansetron with or without a corticosteroid. The primary endpoints in such studies can be the performance status, Quality of Life scores, time to progression of the disease, morbidity, mortality or an increase in the period of progression-free survival. Tumor assessment in the form of dynamic contrast-enhanced Magnetic Resonance Imaging (MRI) is a suitable approach to determine the effect of the treatment.

In one aspect, the present invention provides a commercial package comprising a 4-pyridylmethyl-phthalazine derivative and at least one compound selected from the group consisting of a platinum compound and/or an antineoplastic antimetabolite and, optionally, folinic acid, in which the active ingredients are present in each case in free form or in the form of a pharmaceutically acceptable salt and optionally at least one pharmaceutically acceptable carrier together with instructions for simultaneous, separate or sequential use thereof in the treatment of gastrointestinal, genitourinary, lymphoid or pulmonary (small cell and non-small cell) cancer or cancer of neural crest cell origin in patients having high serum or plasma LDH5 levels.

In a further aspect, the present invention provides a method to diagnose subjects suffering from gastrointestinal, genitourinary, lymphoid or pulmonary (small cell and non-small cell) cancer or a cancer of neural crest origin, especially metastatic colorectal cancer, who may be suitable candidates for the treatment with VEGF-R inhibitors, preferably those of formula I, alone or in combination with chemotherapy, comprising assaying LDH5 levels in a biological sample from said subject, especially a blood or serum or plasma sample, wherein subjects having high serum or plasma LDH5 levels compared to controls would be suitable candidates for treatment with VEGF-R inhibitors alone or in combination with chemotherapy.

### Experimental Part

PTK787/ZK222584 (vascular endothelial growth factor (VEGF) receptor tyrosine kinase inhibitor), Avastin (VEGF-A humanized antibody) and DC101 (rat anti mouse VEGFR2 inhibitor) are used as example compounds to decrease tumor burden and impact serum/plasma levels of LDH5.

### Materials

A zymogram technique from Helena Biosciences is used to separate the LDH isoforms on an agarose gel. Helena biosciences machine SAS-1 Plus (Cat. Nr. 1531). LDH isoenzyme measurements have been estimated using a Diagnostic kit (Cat. Nr. 201300) from Helena BioSciences Europe, Colima Avenue, Sunderland Enterprise Park, Sunderland Tyne & Wear, SR53 XB, England. Cat. No. 210300 Application blades

Cat. No. 210100 Disposable Sample Cups
Cat. No. 5014 Development weight
Cat. No. 4062 Incubation chamber
Cat. No. 3100 Rep Prep buffer
Destain solution: Mix 100 ml of glacial acetic acid and 900 ml of purified water. Store in a tightly stoppered bottle

### Procedure

35 μl plasma samples were pipetted into the disposable sample cups (#210100) and run as described by the kit. Briefly, the sample tray was carefully placed onto the applicator drawer. 400 μl of the Rep Prep buffer (#3100) had been dispensed onto the heat sink and the gel, with the agarose gel side up, was placed carefully onto the heat sink avoiding air bubbles under the gel. The surface of the gel was dried with a blotter C paper to remove buffer excess on top of the gel. Two electrodes were attached onto the top side of the electrode posts so that they contact the gel blocks, the cover was placed over the gel and electrodes and pressed for 5 seconds to ensure contact. One applicator blade assembly (#210300) was placed into the top position on the instrument and the LD electrophoresis was performed. Five samples applications were performed by the machine. Agarose gel was run at 80 volts for 20 minutes at 15° C. Approximately 3-4 minutes before the end of electrophoresis, one vial of LD Isoenzyme reagent were reconstituted by adding 1 ml of LD Isoenzyme diluent. The entire content of the LD Isoenzyme reagent was poured along the middle of the gel. To ensure equal spreading of the reagent, one piece of the reagent spreading film was carefully applied on top of the gel. The gel was then incubated for 25 minutes at 45° C. After incubation the reagent spreading film and both gel blocks were removed. The gel was then destained with 10% acetic acid for 2 minutes. A blotter B was wetted in destain solution and was put on the surface of the gel, followed by three folded paper towels. The gel was then pressed for 5 minutes with the development weight. The gel was scanned immediately after completion. The five isoenzyme bands were quantified with the Alpha Ease v5.5 software program.

### Quantification of LDH Bands

The zymogram gel were scanned using the hp scanjet 5590. The scanned pictures were saved as a TIF file and the bands were quantified using the Microsoft program Alpha Ease V5.5. Values are expressed as integrated density values (IDV=sum of all pixels in the area defined)

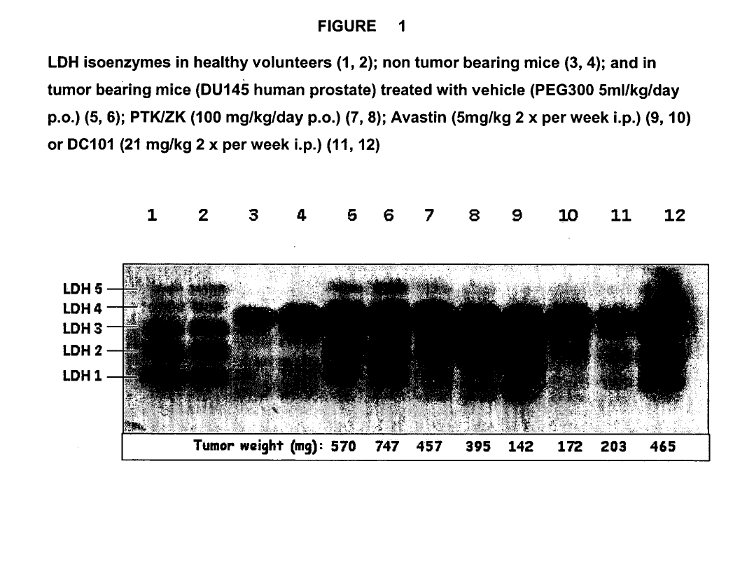

The accompanying drawings (FIG. 1-4), which are incorporated in and constitute a part of the specification, illustrate exemplary embodiments of the present invention.

### EXAMPLE 1

Plasma was taken from 2 normal healthy volunteers, 2 non tumor bearing mice, and 8 mice implanted with a human prostate tumor line, DU145, of which 2 were treated with vehicle (PEG300), 2 with PTK/ZK 100 mg/kg/p.o. per day, 2 with Avastin 5 mg/kg twice per week i.p., and DC101 21 mg/kg i.p. twice per week. DU145 tumors were implanted on day 0 and treatment was from day 17 to 43.

Human LDH isoforms have a different electrophoretic shift than mouse LDH isoenzymes. This is because the M subunit is substantially different between the 2 species. Since LDH5 is composed of only M subunits, this shows the greatest difference between human and mouse (compare FIG. 1; lanes 1 and 2 with 3 and 4). LDH4, 3 and 2 also show differing shifts as they also contain M subunits. The LDH1 bands appear similar between mouse and human. Another startling difference between non tumor bearing mice and humans, is the predominance of the isoforms. Humans have more LDH1, 2, 3 in their plasma, whereas mice have high quantities of LDH5.

One can take advantage of the differing electrophoretic shifts to distinguish human derived LDH (tumor derived) versus mouse LDH (stromal derived) in this DU145 model.

In FIG. 1; lanes 5 and 6 (vehicle) resp. 7 and 8 (PTK/ZK), 9 and 10 (Avastin), 11 and 12 (DC101—an anti mouse VEGFR2 antibody) one can clearly see a human LDH5 band and therefore this must have come from the tumor. This also correlates with tumor burden as shown in milligrams for each tumor at the bottom of the gel of FIG. 1.

There also appears to be a stromal component though this is more difficult to ascertain and quantify as the bands from mouse and human will have run together.

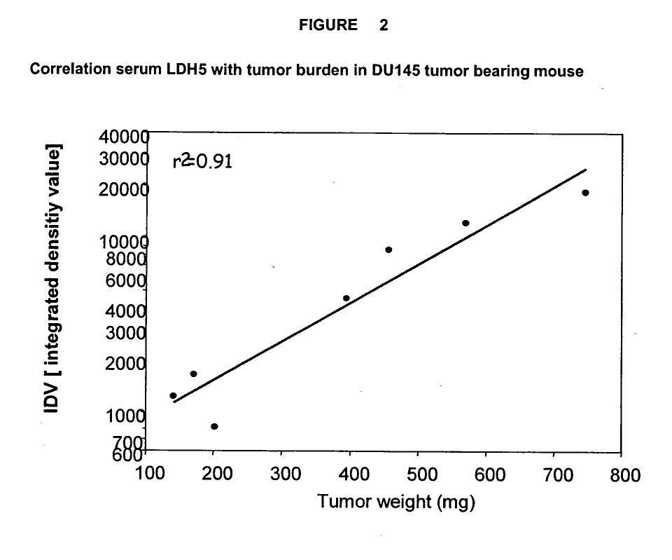

The human LDH5 band was quantified for each of the DU145 bearing mouse plasma samples. In FIG. 2 the density of each band was plotted against the weight of the tumor excised from the animal and shows a good correlation of LDH5 with the tumor burden.

### EXAMPLE 2

Plasma was taken from 2 normal healthy volunteers, 2 non tumor bearing mice, and 8 mice implanted with a human colon tumor line, SW480, treated with vehicle. Tumor was implanted on day 0 and vehicle treated (PEG300) daily from day 7 until day 22 when the animal was sacrificed and plasma taken for analysis.

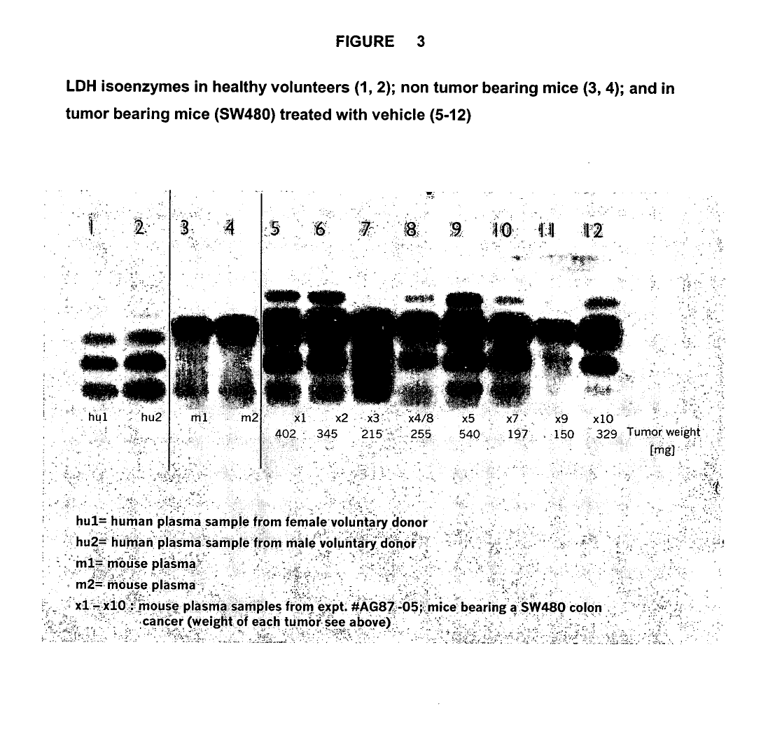

In FIG. 3; lanes 5 to 12 (vehicle), one clearly sees a human LDH5 band and therefore this must have come from the tumor. This also correlates with tumor burden as shown in milligrams for each tumor at the bottom of the gel of FIG. 3. There also appears to be a stromal component though this is more difficult to ascertain and quantify as the bands from mouse and human will have run together.

Therefore in this model, we see that at least some LDH5 is tumor derived and this correlates with the tumor burden.

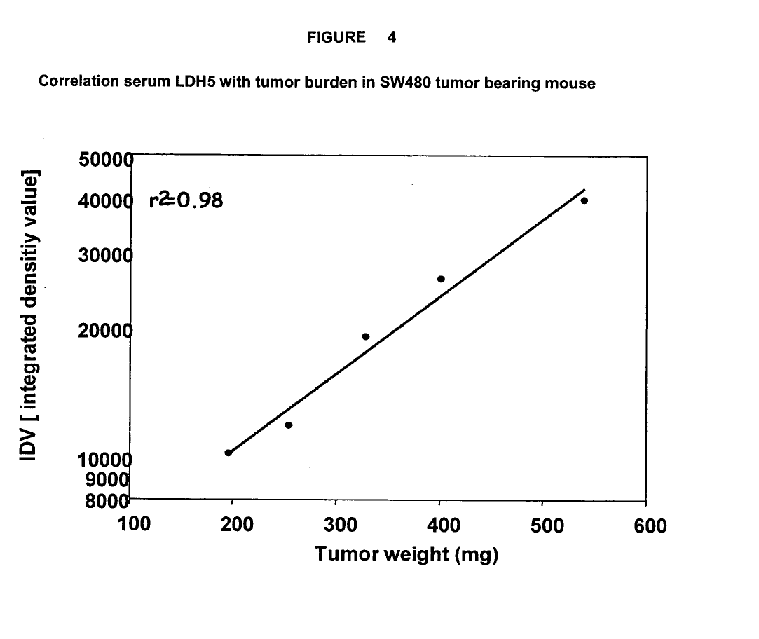

The human LDH5 band was quantified for each of the SW480 bearing mouse plasma samples. In FIG. 4 the density of each band was plotted against the weight of the tumor excised from the animal. In this model, LDH5 correlates with the tumor burden.

### REFERENCES

1. Stokkel M P, Van Eck-Smit B L, Zwinderman A H, Willems L N, Pauwels E K: The diagnostic value of pretreatment serum LDH in patients with limited disease small-cell lung carcinoma. Int J Biol Markers. 1997; 12: 162-7.
2. Koukourakis M I, Giatromanolaki A, Brekken R A, Sivridis E, Gatter K C, Harris A L, Sage E H: Enhanced expression of SPARC/osteonectin in the tumor-associated stroma of non-small cell lung cancer is correlated with markers of hypoxia/acidity and with poor prognosis of patients. Cancer Res. 2003; 63:5376-80.
3. Koukourakis M I, Giatromanolaki A, Sivridis E, Bougioukas G, Didilis V, Gatter K C, Harris A L: Lactate dehydrogenase-5 (LDH-5) overexpression in non-small-cell lung cancer tissues is linked to tumour hypoxia, angiogenic factor production and poor prognosis. Br J Cancer. 2003; 89: 877-85
4. Tas F, Aykan F, Alici S, Kaytan E, Aydiner A, Topuz E: Prognostic factors in pancreatic carcinoma: serum LDH levels predict survival in metastatic disease. Am J Clin Oncol. 2001; 24: 547-50
5. Walenta S, Mueller-Klieser W F: Lactate: mirror and motor of tumor malignancy. Semin Radiat Oncol. 2004; 14: 267-74
6. Fountzilas G, Gossios K, Zisiadis A, Svarna E, Skarlos D, Pavlidis N: Prognostic variable in patients with advanced colorectal cancer treated with fluorouracil and leucovorin-based chemotherapy. Med Pediatr Oncol. 1996; 26: 305-17
7. Koukourakis M I, Giatromanolaki A, Simopoulos C, Polychronidis A, Sivridis E: Lactate dehydrogenase 5 (LDH5) relates to up-regulated hypoxia inducible factor pathway and metastasis in colorectal cancer. Clin Exp Metastasis. 2005; 22: 25-30
8. Sivridis E, Giatromanolaki A, Koukourakis M I: Proliferating fibroblasts at the invading tumour edge of colorectal adenocarcinomas are associated with endogenous markers of hypoxia, acidity, and oxidative stress. J Clin Pathol. 2005; 58: 1033-8
9. Tanaka T, Fujii M, Nishikawa A, Bunai Y, Obayashi F, Sugie S, Shima H, Yoshimi N, Kuniyashu T, Kato K, et al. A cytochemical study of lactic dehydrogenase (LDH) isoenzymes in human lung cancer. Cancer Detect Prev. 1984; 7: 65-71
10. Fujii M, Mori H, Kato K, Takahashi M. Cytochemical studies of LDH isoenzymes in experimental bladder tumors. J Urol. 1982 December; 128:1349-52.
11. Koukourakis M I, Giatromanolaki A, Sivridis E; Lactate dehydrogenase isoenzymes 1 and 5: differential expression by neoplastic and stromal cells in non-small cell lung cancer and other epithelial malignant tumors. Tumour Biol. 2003 August-September; 24(4):199-202.

1: A non invasive method for screening patients for tumor burden by measuring serum or plasma LDH5 for selecting patients for successful therapy with a VEGF-R inhibitor, high serum or plasma LDH5 being a criteria for selecting candidates.

2: A non invasive method for screening patients for tumor burden by measuring serum or plasma LDH5 for selecting patients for successful therapy with a 4-pyridylmethyl-phthalazine derivative, high serum/plasma LDH5 being a criteria for selecting candidates.

3: A non invasive method for screening patients for tumor burden by measuring serum or plasma LDH5 as a surrogate/biomarker of tumor burden for monitoring response or relapse to therapy with a VEGF-R inhibitor.

4: A non invasive method for screening patients for tumor burden by measuring serum or plasma LDH5 as a surrogate or biomarker of tumor burden for monitoring response or relapse to therapy with a 4-pyridylmethyl-phthalazine derivative.

5: A method of treating gastrointestinal, genitourinary, lymphoid or pulmonary (small cell and non-small cell) cancer or a cancer of neural crest origin comprising administering a therapeutically effective amount of a VEGF-R inhibitor to a human patient having high serum or plasma LDH5 levels.

6: A method of treating colorectal cancer comprising administering a therapeutically effective amount of a VEGF-R inhibitor to a human patient having high serum or plasma LDH5 levels.

7: A method of treating gastrointestinal, genitourinary, lymphoid or pulmonary (small cell and non-small cell) cancer or a cancer of neural crest origin comprising administering a therapeutically effective amount of a 4-pyridylmethyl-phthalazine derivative to a human patient having high serum or plasma LDH5 levels.

8: A method of treating colorectal cancer comprising administering a therapeutically effective amount of a 4-pyridylmethyl-phthalazine derivative to a human patient having high serum or plasma LDH5 levels.

9: The method according to claim 2, comprising administering a therapeutically effective amount of a 4-pyridylmethyl-phthalazine derivative of formula I 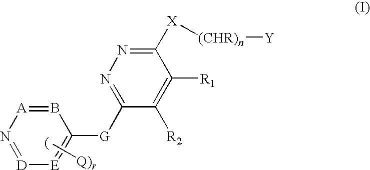
wherein
r is 0 to 2,
n is 0 to 2,
m is 0 to 4,
R₁ and R₂ (i) are lower alkyl or
(ii) together form a bridge in subformula I* 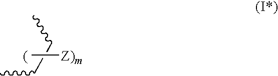
the binding being achieved via the two terminal carbon atoms, or
(iii) together form a bridge in subformula I** 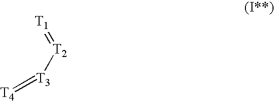
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl,
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group,
to a warm-blooded animal in need thereof.

10: Method of claim 9 wherein the 4-pyridylmethyl-phthalazine derivative of formula I is PTK/ZK.

11: The method of claim 10 wherein between 1200 mg/day to 1300 mg/day of PTK/ZK is administered.

12: The method of claim 10 wherein 1250 mg/day of PTK/ZK is administered.

13-21. (canceled)

22: A commercial package comprising a 4-pyridylmethyl-phthalazine derivative and at least one compound selected from the group consisting of a platinum compound and/or an antineoplastic antimetabolite and, optionally, folinic acid, in which the active ingredients are present in each case in free form or in the form of a pharmaceutically acceptable salt and optionally at least one pharmaceutically acceptable carrier together with instructions for simultaneous, separate or sequential use thereof in the treatment of gastrointestinal, genitourinary, lymphoid or pulmonary (small cell and non-small cell) cancer or a cancer of neural crest origin, especially colorectal cancer, in patients having high serum or plasma LDH5 levels.

23: A method of diagnosing subjects suffering from gastrointestinal, genitourinary, lymphoid or pulmonary (small cell and non-small cell) cancer or a cancer of neural crest origin, especially colorectal cancer, who may be suitable candidates for the treatment with VEGF-R inhibitors alone or in combination with chemotherapy, comprising assaying LDH5 levels in a biological sample from said subject, wherein subjects having high serum or plasma LDH5 levels compared to controls would be suitable candidates for treatment with VEGF-R inhibitors alone or in combination with chemotherapy.
